(19) United States
(12) Patent Application Publication
(10) Pub. No.: US 2005/0025776 A1
(43) Pub. Date: Feb. 3, 2005

(54) JAM-1 as ligand of the beta-2 integrin LFA-1

(76) Inventor: Christian Weber, Aachen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/411,397

(22) Filed: Apr. 11, 2003

Related U.S. Application Data
(60) Provisional application No. 60/371,734, filed on Apr. 12, 2002.

(51) Int. Cl. A61K 48/00; A61K 39/00

(52) U.S. Cl. 424/185.1; 514/44

(57) ABSTRACT

Inflammatory recruitment of leukocytes is governed by dynamic interactions of integrins with endothelial immunglobulin superfamily (IgSF) proteins. We have identified the IgSF member junctional adhesion molecule-1 (JAM-1) as a ligand of the β₂ integrin lymphocyte function-associated antigen-1 (LFA-1). Under static and physiologic flow conditions, JAM-1 contributed to LFA-1-dependent transendothelial migration of T cells and neutrophils, and also to LFA-1-mediated arrest of T cells triggered by chemokines on endothelium co-stimulated with cytokines to re-distribute JAM-1 from the tight junctions. Transfectants expressing JAM-1 supported LFA-1-mediated adhesion of leukocytes which required the membrane-proximal Ig-like domain 2 of JAM-1. Thus, JAM-1 is a counter-receptor for LFA-1 ideally situated to guide and control transmigration during leukocyte recruitment.

This application claims the benefit of the filing date of U.S. Provisional Application No. 60/371,734 filed Apr. 12, 2002.

### DESCRIPTION

The present invention relates to a method of modulating lymphocyte function-associated antigen-I (LFA-1)-mediated processes or disorders by modulating, e.g. inhibiting or stimulating the interaction between LFA-1 and junctional adhesion molecules (JAM-1)

Leukocyte emigration plays a central role in inflammatory and immune responses and is regulated by the sequential involvement or various signal and adhesion molecules (1). Exposure of leukocytes to chemokines released and presented by inflamed tissues can trigger the differential activation of β₁ and β₂ integrins which has been implicated in coordinating distinct steps of leukocyte extravasation, for example initial arrest versus subsequent transendothelial migration (2). Of the β₂ integrin subfamily, a transient upregulation of lymphocyte function-associated antigen-1 (LFA-1, α_{L}β₂)-dependent adhesion by chemokines has been involved in the firm arrest of lymphocytes in shear flow (3). Moreover, a dynamic regulation of LFA-1 avidity appears to be required for transendothelial diapedesis of mononuclear cells (4,5). The known ligands of LFA-1, intercellular adhesion molecule-1 (ICAM-1), ICAM-2 and ICAM-3 belong to the immunoglobulin superfamily (IgSF) (6-9). Whereas ICAM-1 and ICAM-2 expressed on endothelium have been implicated in leukocyte adhesion or transendothelial migration, ICAM-3 is exclusively present on leukocytes where it may augment LFA-1- and ICAM-1-mediated adhesion and signaling by inducing clustering and redistribution of LFA-1 via low affinity interactions (6-10). Additional examples of integrin interactions with IgSF members that contribute to adhesion or transmigration of leukocytes include α_{M}β₂ (Mac-1) with ICAM-1, α₄β1 (VLA-4) with vascular cell adhesion molecule-1 (VCAM-1) α₄β₇ with mucosal addrassin call adhesion molecule-1 (MAd-CAM-1) and αᵥβ₃ with platelet endothelial cell adhesion molecule-1 (PE-CAM-1) (11-14).

In a yeast two-hybrid search for interaction partners of LFA-1, we identified junctional adhesion molecule-1 (JAM-1), a IgSF member consisting of two Ig-like domains, which is expressed predominantly at tight junctions of endothelial or epithelial cells (15-20). JAM-1 participates in the contact of neighboring cells by homophilic interactions and is redistributed from junctions by certain proinflammatory cytokines. Like other IgSF proteins, JAM-1 has also been found to be expressed on hematopoietic cell types.

A blocking JAM-1 monoclonal antibody (mAb), BV11, inhibited transendothelial migration of monocytes without affecting homophilic JAM-1 binding, as reflected by the paracellular permeability of JAM-1 transfectant monolayers for dextran (15,23). Since the murine monocytes used these studies do not express surface JAM-1, this suggests a role of JAM-1 in the control of transmigration involving a yet to be identified heterophilic interaction partner on leukocytes. On the other hand, the effects of BV11 may be due to an inhibition of dimerization (54). Like other IgSF integrin ligands, for example PECAM-1 (24), JAM-1 may be ideally situated to serve as a junctional gate keeper promoting the transmigration of leukocytes.

Since LFA-1 is known to be crucial for leukocyte transmigration, we tested, if JAM-I may be involved in leukocyte diapedesis. We provide the first evidence that JAM-1 is a LFA-1 ligand which binds its counter-receptor via its membrane-proximal domain 2 and thereby plays a role in sequential steps of adhesion and subsequent transmigration during recruitment of memory T cells and neutrophils. These results suggest a role for JAM-1 in the regulation of LFA-1 mediated processes or disorders.

Thus, a subject matter of the invention is a method of monitoring or modulating lymphocyte function-associated antigen-1 (LFA-1) mediated processes or disorders comprising contacting a cell or an organism, which expresses LFA-1 with (i) a polypeptide selected from junctional adhesion molecule-1 (JAM-1) or an active fragment thereof, (ii) a nucleic acid molecule encoding the polypeptide of (i) or (iii) an effector of the polypeptide of (i) or the nucleic acid of (ii). The polypeptide (i) is preferably a mammalian JAM-1, which may be selected from human JAM-1 (GenBank Accession No. AF172398) or mouse JAM-1 (GenBank Accession No. U89915) or active fragments thereof. The active fragment preferably comprises the membrane-proximal domain 2 of JAM-1 which is sufficient to mediate adhesion to LFA-1, e.g. human LFA-1 (GenBank Accession No.

NM _{—}002209 for αL). More preferably, active fragments of JAM-1 comprise a segment of domain 2-neighbouring domain 1, particularly the amino acids 125-212 of human JAM-1 or a part thereof. The JAM-1 fragment may be a soluble protein or a peptide having a length of preferably at least 4 amino acids, more preferably at least 5 amino acids and most preferably at least 25 amino acids. Further, the JAM-1 fragment may be a fusion protein, particularly a soluble fusion protein wherein a soluble JAM-1 fragment may be coupled to an effector group, or a marker group, e.g. an IgG-Fc portion or a FLAG epitope.

The nucleic acid (ii) may encode a mammalian JAM-1, preferably selected from a human JAM-1 or active fragments thereof, as described above. More preferably, the nucleic acid is selected from human JAM-1 cDNA (GenBank Accession No. AF172398 or WO98/24897) and/or a sequence complementary thereto. Further, the invention relates to JAM-1 related sequences which hybridize under stringent conditions to a JAM-1 sequence as described above. Stringent condition means that after washing for 1 h with 1×SSC and 0.1% SDS at 50° C., preferably at 55° C., more preferably at 62° C., and most preferably at 68° C., particularly for 1 h and 0.2×SSC and 0.1% SDS at 50° C., preferably at 55° C., more preferably at 62° C. and most preferably at 68° C., a positive hybridization signal is observed.

The nucleic acid may encode a JAM-1 fragment, particularly a soluble polypeptide as described above. The nucleic acid may be operatively linked to an expression control sequence, particularly an expression control sequence allowing gene expression in eukaryotic organisms, particularly, mammals. Examples of suitable expression control sequences comprising promoter, enhancer and, optionally, translation start signal sequences are known in the art. The expression vector is preferably a gene transfer vector, e.g. a non-viral vector, such as a plasmid or a viral vector, which allows the transfection or infection of suitable host cells or host organisms, particularly eukaryotic cells or organisms, e.g. human cells or human organisms.

The effector (iii) of JAM-1 is preferably capable of monitoring or modulating, e.g. inhibiting or stimulating the interaction of JAM-1 and LFA-1. For diagnostic purposes the effector may be coupled to a labelling group as known in the art. In a preferred embodiment the effector interacts with JAM-1, particularly with the membrane-proximal domain 2 of JAM-1. In a further preferred embodiment the effector may interact with LFA-1, particularly with the inserted (I) domain, preferably comprises (amino acids 125-321 and more preferably comprising amino acids 237-247). The interaction may be direct, e.g. by specific binding or indirect, e.g. by binding to a substrate or by altering gene expression.

The effector may be selected from antibodies, e.g. polyclonal antibodies, monoclonal antibodies, chimeric antibodies, humanized antibodies, human antibodies, recombinant antibodies, such as single-chain antibodies and fragments thereof. Especially preferred is a blocking antibody against human JAM-1. These antibodies may be obtained from mice immunized with JAM-1 comprising polyclonal sera capable of inhibiting transendothelial migration of human neutrophils and/or memory T cells.

From spleen cells of these mice, which may be fused with a myeloma cell, wherein a monoclonal antibody producing hybridoma cell is obtained.

Monoclonal antibodies may be chimerized or humanized or otherwise modified according to known methods.

Further, the effector may be a biologically active nucleic acid, e.g. an antisense molecule capable of modulating the expression of JAM-1 or LFA-1. Furthermore, the biologically active nucleic acid may be an RNA interference molecule or a ribozyme. Methods of manufacturing suitable antisense molecules, RNAi molecules or ribozymes are known in the art.

in a different embodiment, the effector may be a peptide or a low molecular weight organic molecule. Suitable peptides may be derived from the JAM-1 proximal membrane domain 2, preferably from amino acids 125-212 including the c′-c″ hairpin Ala175-Lys179 and/or the linker sequence Val127-Val129 or the inserted (I) domain of LFA-1, preferably from amino acids 125-321 and more preferably from amino acids 237-247. The peptide preferably has a length of from 4-25 amino acids, more preferably from 5-15 amino acids. The peptide may comprise genetically encoded amino acids and/or non-genetically encoded amino acids including D-amino acids and 13-amino acids. The effector may also be a cyclic peptide, a peptide mimetic or a scaffold structure, which may be designed from a peptide according to known methods. Suitable low molecular weight organic molecules may be identified and characterized by screening procedures from libraries as described below.

The invention also relates to host cell or a non-human host organism capable of overexpressing JAM-1 and LFA-1 or active fragments thereof. The non-human host organism may be a transgenic animal which is used as a system for analyzing JAM-1/LFA-1 interactions or for performing screening procedures in order to identify and/or characterize JAM-1 effector molecules.

Further, the invention relates to a cell-free assay system comprising a JAM-1 polypeptide and/or LFA-1 polypeptide or active fragments thereof. Particularly preferred is a soluble recombinant JAM-1 fusion protein. The cell-free system may also be used as a system for performing screening procedures in order to identify or characterize JAM-1 effector molecules.

Still a further embodiment of the invention relates to a method for identifying and/or characterizing the effect of compounds on LFA-1 mediated processes or disorders comprising:
(a) providing an assay system comprising
   (i) a JAM-1 polypeptide or an active fragment thereof or a host cell or a non-human host organism capable of overexpressing JAM-1 or an active fragment thereof and
   (ii) a LFA-1 polypeptide or an active fragment thereof or a host cell or a non-human host organism capable of overexpressing LFA-1, or an active fragment thereof,
(b) contacting the system of (a) with a compound to be tested and
(c) determining the effect of the compound on the interaction between (i) and (ii).

A compound which is capable of modulating the interaction between JAM-1 and LFA-1, and which has been identified and/or characterized by the method as described above, or a compound derived therefrom, e.g. by molecular modification or modelling, may be formulated as a pharmaceutical composition.

The term “LFA-1 mediated processes or disorders” as used in the present application relates to any physiological process or disorder, which is associated with or caused an activity of LFA-1, particularly by an abnormal, e.g. decreased or increased, activity. Preferably, the invention relates to the monitoring or modulating of processes or disorders which comprise a cell migration, e.g. a transendothelial migration of leukocytes, such as T cells and neutrophils. Further, the processes or disorders may comprise an arrest of T cells, particularly a T cell arrest triggered by chemokines. The present invention is particularly suitable for the diagnosis, prevention or Treatment of infectious or inflammatory disorders associated with transendothelial migration of leukocytes, such as vascular inflammatory disorders such as artherosclerosis, arteritis or reperfusion injury, autoimmune is disorders, connective tissue disorders such as rheumatoid arthritis, disorders of the nervous system such as meningitis or multiple sclerosis and inflammatory bowel disorders such as ulcerative colitis and Crohn's disease.

The monitoring or modulating of LFA-1 mediated processes or disorders may be effected by administering a pharmaceutical composition comprising the active agent and at least one physiologically acceptable carrier, diluent and/or adjuvant. The composition may be a therapeutic or a diagnostic composition, which contains the active agent in a diagnostically or therapeutically effective amount. More preferably, the pharmaceutical composition is manufactured for human medicine.

The pharmaceutical composition may be administered by any number of routes, e.g. oral, parenteral, transdermal, topical, mucosal, inhalative or rectal means. For example, the administration of proteins and peptides may be accomplished by intraperitoneal or subcutaneous or intraveneous injection or other delivery systems, e.g. as reviewed in (49). The administration of nucleic acids may be accomplished by protocols as described for viral or non-viral gene delivery vectors (50,51). The administration of antibodies and antibody fragments may be accomplished by protocols as described for established therapies using chimeric or humanized antibodies or antibody fragments (52,53). Suitable doses and administration protocols may be determined by the skilled person depending on the type or the variety of the disease or disorder to be treated or prevented.

Further, the invention is explained in more detail by the following figures and examples.

### FIGURE LEGENDS

FIG. 1

Expression of JAM-1

(a) Flow cytometry analysis of JAM-1 surface expression in CHO cells.

Wild-type (WT), vector- and JAM-1-transfected CHO cells were stained with mouse anti-JAM-1 Ab (bold line) or pre-immunization serum (dotted line) and analyzed by flow cytometry. Shown are representative histograms. (b) Immunoblot analysis of JAM-1 protein in CHO cells. Membrane and cytosolic fractions of wild-type or JAM-1-transfected CHO cell lysates were separated by SDS-PAGE. JAM-1 detected as a single band at approximately 40 kDa in a representative blot is indicated by the arrow. (c) Expression of native JAM-1 on HUVEC, HeLa cells, myeloid HL-60 cells, Jurkat and J-β₂.7 T lymphoid cells, and CD4⁺CD45RO⁺ T cells. Cells were stained with JAM-1 Ab (bold line) or pre-immunization serum (dotted line) and analyzed by flow cytometry. Shown are representative FACS histograms.

FIG. 2

JAM-1 Serves as Ligand for LFA-1-Bearing Jurkat T-cells and Purified LFA-1

(a) Adhesion of PMA-stimulated Jurkat cells to adherent, vector- or JAM-1-transfected CHO cell monolayers. (b) Characterization of purified LFA-1. (c) Adhesion of CHO transfectants to immobilized LFA-1. (a, c) Some substrates or cells were pre-incubated without (control) or with blocking β₂ mAb TS1/18 or JAM-1 Ab. After a 30 min incubation, nonadherent cells were removed and adhesion was measured as percentage of input cells. Background binding of unstimulated Jurkat cells was negligible and subtracted from PMA-stimulated binding. Isotype control and preimmunization serum had no significant effect on adhesion (not shown). Data are given as the mean ±SD of 4 separate experiments performed in triplicate. (b) Purified LFA-1 at the concentration used for coating was separated by SDS-PAGE. Western blot analysis with α_{L} or β₂ mAb (left) and silver staining (right) revealed distinct bands at approximately 180 kDa and between 100 and 120 kDa, corresponding to the a and 1 subunits of LFA-1, while JAM-1 was not detectable by western blotting in the same preparation (middle).

FIG. 3

Interaction with JAM-1 is Specific for the β₂ Integrin LFA-1 But Not Mac-1

(a) Adhesion of PMA-stimulated Jurkat and J-β₂.7 cells to adherent, vector- or JAM-1-transfected CHO cell monolayers, (b) Adhesion of PMA-stimulated J-β₂.7-LFA-1 (α_{L}) and J-β₂.7-Mac-1 (α_{M}) transfectants to adherent, vector- or JAM-1-transfected CHO cell monolayers. (c) Adhesion of PMA-stimulated myeloid HL-60 cells on adherent, vector- or JAM-1-transfected CHO cell monolayers. Cells were pre-treated without (control) or with blocking β₂ mAb TS1/18 (a) or α_{L} mAb TS1/22 (c). After a 30 min incubation, non-adherent cells were removed and adhesion was measured as percentage of input cells. Background binding of unstimulated Jurkat, J-β₂.7 or HL-60 cells was negligible and subtracted from PMA-stimulated binding. Isotype control had no significant effect on adhesion (not shown). Data are given as the mean ±SD of 4 separate experiments performed in triplicate.

FIG. 4

Expression of JAM-1 Domain Deletion Mutants in CHO Cells

(a) Schematic illustration of the full-length human JAM-1 protein (aa1-299) and its domain deletion mutants, The N-terminal signal peptide (aa1-27) is marked in grey the predicted transmembrane region (aa236-260) in black. Shown are JAM-1^{ΔD1} lacking the N-terminal domain 1 and JAM-1^{ΔD2} lacking the membrane-proximal domain 2. (b) Verification of wild-type or mutant JAM-1 (without HA epitope tag) expression in CHO transfectants by RT-PCR. PCR with plasmid DNA served as a positive control (PC; 900 bp), is reaction mix without template as a negative control (NC). The upper agarose gel shows PCR products using specific primers for JAM-1, reflecting mRNA expression of full-length JAM-1, JAM-1^{ΔD1} (660 bp) or JAM-1^{ΔD2} (672 bp). The lower gel shows expression of b-actin mRNA (446 bp) as an internal control. (c) Analysis of wild-type or mutant JAM-1 (with a C-terminal HA epitope tag) expression in CHO transfectants by flow cytometry after staining with a polyclonal HA Ab (bold line) or isotype control (dotted line). Shown are representative gels and histograms.

FIG. 5

The Membrane-Proximal Domain 2 of JAM-1 Mediates Interactions with LFA-1 (a, c) Adhesion of PMA-stimulated Jurkat cells to adherent CHO cells. (b, d) Adhesion of CHO cell transfectants to immobilized LFA-1 (b, d). CHO cell transfectants expressed wild-type JAM-1 or the domain deletion mutants JAM-1^{ΔD1} and JAM-1^{ΔD2} without (a, b) or with (c, d) a C-terminal HA epitope tag, as indicated. Non-adherent cells were removed and adhesion was measured as percentage of input cells. Background binding of resting Jurkat cells was substracted from PMA-stimulated binding. Isotype control had no significant effect on binding (data not shown). Data are given as the mean ±SD of 4 separate experiments performed in triplicate.

FIG. 6

JAM-1 Participates in LFA-1-Dependent Adhesion and Transmigration of T Cells on Endothelium Triggered by PMA or SDF-1α Under Static or Physiologic Flow Conditions

(a, b) Adhesion of PMA-stimulated CD4⁺CD45RO⁺ T cells on unstimulated HUVEC (a) or HUVEC co-stimulated with TNF-α and IFN-γ (b). (c) Transendothelial chemotaxis of CD4⁺CD4⁺RO⁺ T cells across unstimulated HUVEC induced by SDF-1α. Adhesion and transmigration were expressed as percentage of input. (d, e) Arrest of CD4⁺CD45RO⁺ T cells triggered by SDF-1a immobilized on HUVEC stimulated with TNF-α (d) or co-stimulated with TNF-α and IFN-γ (e) in flow at 1.5 dyne/cm². After 5 min, firmly adherent T cells were counted and expressed as cells/mm². (f) Spreading and transmigration of CD4⁺CD45RO⁺ T cells induced by SDF-1α immobilized on HUVEC costimulated with TNF-α and IFN-γ at 1.5 dyne/cm². Cells undergoing shape change indicative of spreading or transmigration were analyzed and expressed as % of adherent cells. T cells were pre-treated with LFA-1 mAb TS1/22 and HUVEC were pre-treated with ICAM-1 mAb and/or JAM-1 Ab, as indicated (a-f). Isotype control and pre-immunization serum had no significant effects, while ICAM-1 Fab was similarly effective as ICAM-1 mAb (data not shown), Data are given as mean ±SD representative of at least 3 experiments performed in triplicate. * indicates p<0.05, ** indicates p<0.01.

FIG. 7

JAM-1 Contributes to LFA-1-Mediated Transmigration But Not to Arrest of Neutrophils

(a) Arrest of neutrophils triggered by CXCR2 on HUVEC co-stimulated with TNF-α and IFN-γ in flow at 1.5 dyne/cm². After 5 min, firmly adherent neutrophils were counted and expressed as cells/mm². (b) Transendothelial migration of neutrophils across unstimulated HUVEC induced by IL-8.

Transmigration was expressed as percentage of input. Neutrophils were pre-treated with α_{L} or β₂ mAb and HUVEC were pre-treated with ICAM-1 mAb and/or JAM-1 Ab, as indicated (a, b). Isotype control and pre-immunization serum had no significant effects (data not shown). Data are given as mean ±SD representative of at least 3 experiments performed in triplicate. * indicates p<0.05, ** indicates p<0.01.

### EXAMPLE

1. Materials and Methods

1.1 Expression Vectors

Human JAM-1 cDNA was obtained performing a Gal4-based yeast two-hybrid screening of a human leukocyte cDNA library (Clontech, Palo Alto, Calif.) for interaction partners of the LFA-1 α_{L} subunit. The prey library vector isolated from a yeast colony positive for expressing a LFA-1 interacting protein contained the full-length cDNA of human JAM-1. The coding sequence was amplified by polymerase chain reaction (PCR) with primers 5′-GCGGGGTACCATGGGGACAAAGGCGCAAGTCGA-3′and 5′-GCGGGGATCCTCACACCAGGAATGACGAGGTCT-3′ (JM102) and ligated into the KpnI/BamHI site of the vector pcDNA3.1 (Invitrogen, Groningen, Netherlands). The JAM-1 cDNA constructs lacking domain 1 or 2, JAM^{ΔD1} and JAM^{ΔD2}, respectively, were generated using the PCR overlap extension method. Following primers were used for pJAMDD1 (Dnt124-393): 5′-GCGGGGTACCGCCGCCAGCCA TGGGGACAAAGGCGCAAGTCGA-3′ (JM181) and 5′-GGATGTTAACTGTAGGCTTGGAT GGAGGAATTCTGACTTCAGGTTCAG-3′ to amplify the 5′-fragment of JAM^{ΔD1} and 5′-TTCTGAACCTGAAGTCAGAATTCCTCCATCCAAGCCTACAGTTAACAT-3′ and JM102 to amplify the 3′-fragment. The overlap extension resulting in the spliced 660 bp hybrid fragment JAM^{ΔD1} was subcloned in pcDNA3.1. pJAM^{ΔD2} (Dnt394-650) was generated by fusing two PCR fragments, the first being amplified using the primers JM181 and 5′-CATTTGAAGTCATGGG TGTCCCATAAGGCACAAGCACCATGAGCTTG-3′, the second being amplified with the primers 5′-GGTCAAGCTCATCGTGCTTGTGCCTTATGGGACACCCATGACTTCAAA-3′ and JM102, resulting in the 672 bp hybrid DNA. In addition, all constructs were also adjoined to a C-terminal hemagglutinin (HA)-epitope tag by using the primer JM102HA with the sequence 5′-GCGGGGATCCTCAAGCGTAATCTGGAACATCGTATGGGTATCCAGCCACCAGGAATGACGAGGTCT-3′ instead of JM102. All inserts were sequenced to exclude mismatches.

1.2 Cell lines, Cells, Transfection, Antibodies and Reagents

CHO cells and HeLa cells were cultured in DMEM/F12 medium (Invitrogen) supplemented with 10% heat-inactivated fetal calf serum (FCS) and gentamicin (50 mg/l). Stable transfectants were generated by electroporating 10⁷ cells with 30 mg of cDNA (vector, full-length human JAM-1, pJAMDD1, pJAM^{ΔD2}) and selected with G418 (Calbiochem, La Jolla, Calif.). Jurkat T cells and HL-60 cells (ATCC, Manassas, Va.), α_{L}-deficient Jurkat J-β₂.7 mutants (25), and stable J-β₂.7 transfectants expressing LFA-1 (J-β₂.7-α_{L}) or Mac-1 (J-β₂.7-α_{M}) (26) were maintained as described. HUVEC and Mono Mac 6 cells were grown as described (44). Peripheral blood mononuclear cells and neutrophils were prepared by Ficoll-hypaque density gradient centrifugation and hypotonic erythrocyte lysis, and CD4⁺CD45RO⁺ T cells were isolated by negative selection using magnetic separation (Miltenyi Biotec, Bergisch Gladbach. Germany) (45). Polyclonal anti-JAM-1 serum was generated by DNA immunization (46) of mice with full-length human JAM-1 cDNA (Eurogentec, Herstal, Belgium) and tested in comparison to pre-immunization serum. Hybridoma cells expressing α_{L} mAb TS1/22 or β₂ mAb TS1/18 were purchased from ATCC (1016121RE and 1016125RE). ICAM-1 mAb or Fab fragment RR1/1 and VLA-4 mAb HP1/2 were kindly provided by Dr. R. Rothlein (Boehringer Ingelheim) and Dr. M. Hemler (Harvard Medical School), respectively. Mouse polyclonal Ab to HA (Y-11) and goat polygonal Ab to α_{L} (C17) were from Santa Cruz (Santa Cruz, Calif.), mouse mAb to β₂ (MEM48) was from R&D Systems (Wiesbaden, Germany). Human recombinant TNF-α, IFN-γ, SDF-1α and IL-8 were from PeproTech (Rocky Hill, N.J.). Reagents were from Sigma Co (Deisenhofen, Germany) or Merck (Darmstadt, Germany) unless specified.

1.3 Western Blot, Flow Cytometry and RT-PCR

Membrane and cytosolic extracts were prepared from cells homogenized in hypotonic lysis buffer including complete protease inhibitor cocktail (Roche, Basel, Switzerland) by ultracentrifugation, and membrane fractions were dissolved in lysis buffer with 1% Igepal CA-630, For immunoblotting, 20 mg of fractions or 10 ml of purified LFA-1 were separated by SDS-PAGE and transferred to nitrocellulose membranes, blocked with 5% non-fat dry milk (Bio-Rad, Hercules, Calif.), incubated with JAM-1 Ab, HA Ab, β₂ mAb or α_{L} MAb for 1 h at RT and detected with HRP-conjugated anti-mouse or anti-goat Ig (Amersham, Uppsala, Sweden) and enhanced chemiluminescence (Pierce, Rockford, Ill.). Purified LFA-1 separated by SDS-PAGE was also detected by silver staining For flow cytometry, cells were incubated with specific Ab, pre-immunization serum or mouse IgG isotype control for 30 min on ice, reacted with FITC-conjugated goat anti-mouse IgG, and analyzed in a FACScan (Becton Dickinson, San Jose, Calif.). For FACS analysis of C-terminal HA epitope expression, cells were permeabilized by treatment with 0.2% triton-X for 2 min. For RT-PCR, total RNA from 6×10⁶ transfectants was isolated using Trizol (Invitrogen). After DNase treatment, cDNA was synthesized from 5 mg of RNA with Superscript 11 reverse transcriptase (Invitrogen) and used for PCR amplification with appropriate primers.

1.4 Cell Adhesion Assay

Static cell adhesion assays were performed using 96-well microtiter plates (ICN, Costa Mesa, Calif.), as described (2, 26). Adherent CHO cells or purified LFA-1 obtained by immunoaffinity chromatography from organs (42) immobilized at 3 mg/ml in 10 mM Tris pH 9.0 overnight were blocked by incubation with HHMC (Hank's balanced salt solution, 1 mM CaCl₂, 1 mM MgCl₂, 10 mM HEPES) containing 0.5% bovine serum albumine (BSA) for 2 h. Cells in suspension subjected to adhesion were labeled with the fluorescent dye 2′,7′-bis-(2-carboxyethyl)-5-(or-6)-carboxy-fluorescein-acetoxymethylester (BCECF-AM; Molecular Probes, Eugene, Oreg.) at 1 mg/ml for 30 min. 10⁵ cells in 50 ml HHMC per well were allowed to adhere for 30 min at 37° C. Jurkat or HL-60 cells were stimulated with PMA (100 ng/ml) or SDF-1α (1 μg/ml). Some cells were pre-incubated with TS1/10 mAb (1:2 dilution of hybridoma supernatant) or JAM-1 Ab (1:50). Non-adherent cells were removed by a flick wash. The fluorescence of input and adherent cells was determined with a fluorescence plate reader (SpectraFluor Plus, Tecan, Crailsheim, Germany).

1.5 Static Leukocyte Adhesion and Transmigration Assays

HUVEC were grown on 5 mm Transwell-filter inserts (Costar, Corning Inc., Acton, Mass.) or 96-well microtiter plates, as described (5,47) Some HUVEC were pre-incubated with JAM-1 Ab or pre-immunization serum (1:50 dilution), ICAM-1 mAb or Fab RR1/1, or isotype control, some T cells and neutrophils (pre-treated with 5% human serum to block Fc-receptors) were pre-incubated with LFA-1 mAbs TS1/22 (α_{L}), TS1/118 (β₂) or isotype control (all 10 μg/ml). CD4⁺CD45RO⁺ T cells and neutrophils in RPMI-1640 with 0.5% BSA were allowed to transmigrate at 37° C. towards SDF-1α (1 μg/ml) for 90 min or towards IL-8 (100 ng/ml) for 60 min, respectively. Transmigrated cells and input were counted by flow cytometry with standard beads. CD4⁺CD45RO⁺ T cells (10⁵ cells in 50 ml HHMC with 0.5% BSA) labeled with BCECF-AM and stimulated with SDF-1α (1 μg/ml) were allowed to adhere for 5 min at 37° C. Non-adherent cells were removed by multiple flick washes, and the fluorescence of input and adherent cells was determined. Assays on HUVEC pre-treated with TNF-α (100 U/ml) and IFN-γ (200 U/ml) were performed after pre-incubation of T cells with VLA-4 mAb (10 μg/ml). Transmigration and adhesion of CD4⁺CD45RO⁺ T cells or neutrophils was expressed as percentage of input.

1.6 Leukocyte Adhesion and Transmigration Assays Under Flow Conditions

Laminar flow assays were performed as described (44,45,48). Confluent HUVEC were pre-incubated with SDF-1α (1 μg/ml), JAM-1 Ab or pre-immunization serum (1:50 dilution), ICAM-1 mAb or Fab RR1/1, or isotype control (10 μg/ml) for 30 min and inserted in a parallel wall flow chamber. T cells or neutrophils pretreated with 5% human serum to block Fc receptors (5×10⁵/ml each) in HHMC were perfused at 1.5 dyne/cm² after pre-treatment with TS1/22 mAb or isotype control (10 μg/ml) for 20 min. Assays on HUVEC pre-treated with TNF-α (100 U/ml) and IFN-γ (200 U/ml) were performed after pre-incubation of T cells with VLA-4 mAb (10 μg/ml). Cell arrest and spreading were analyzed in multiple high power fields recorded by video microscopy.

1.7 Statistical Analysis

Statistical analysis was performed with InStat software (GraphPad Software, San Diego, Calif.), using analysis of variance (ANOVA) and signed-rank tests according to Kruskal-Wallis as multiple comparison procedures, and Tukey tests, Student's t-tests and Bonferroni tests to determine the statistical significance between two interventions, where appropriate. A stastistically significant difference for a particular comparison was only claimed when the criterion p<0.05 or better was fulfilled. Similarly, the effect of a JAM-1 and ICAM-1 Ab combination was only considered as additive when the level of significance as indicate by the p-value was lower for the combination than for either antibody alone and the combination was statistically different with p<0.05, as compared to either intervention alone.

2. Results

2.1 Expression of JAM-1 in Transfectants and Various Cell Types

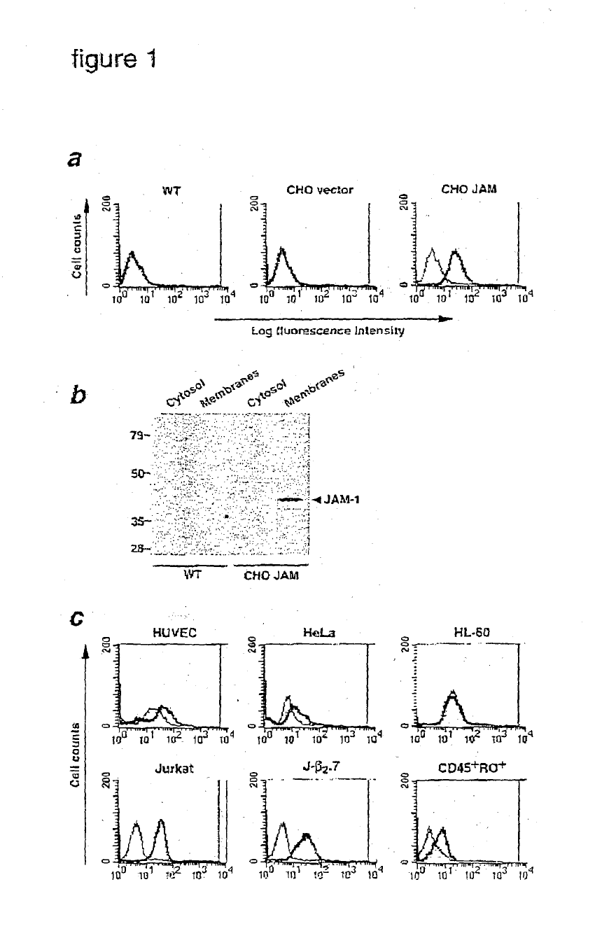

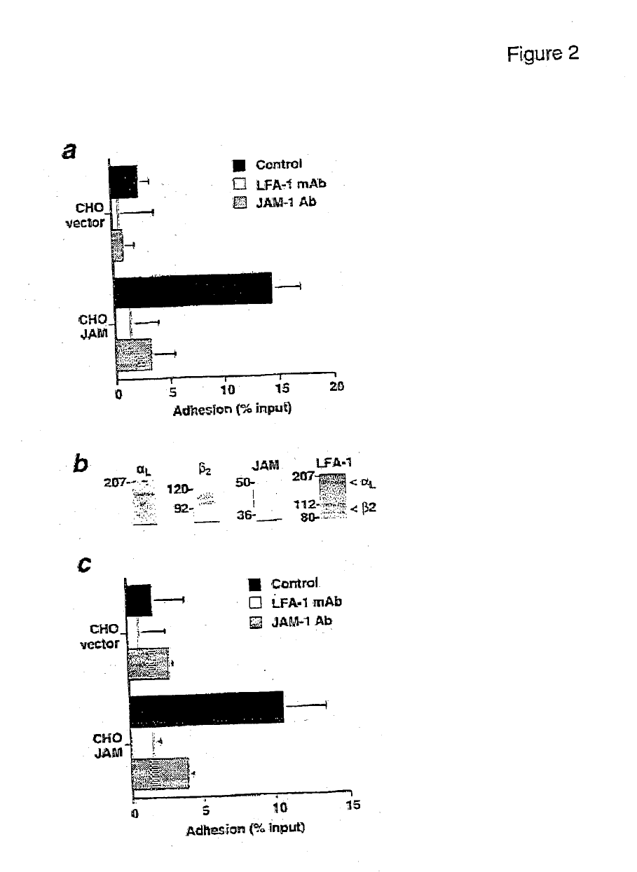

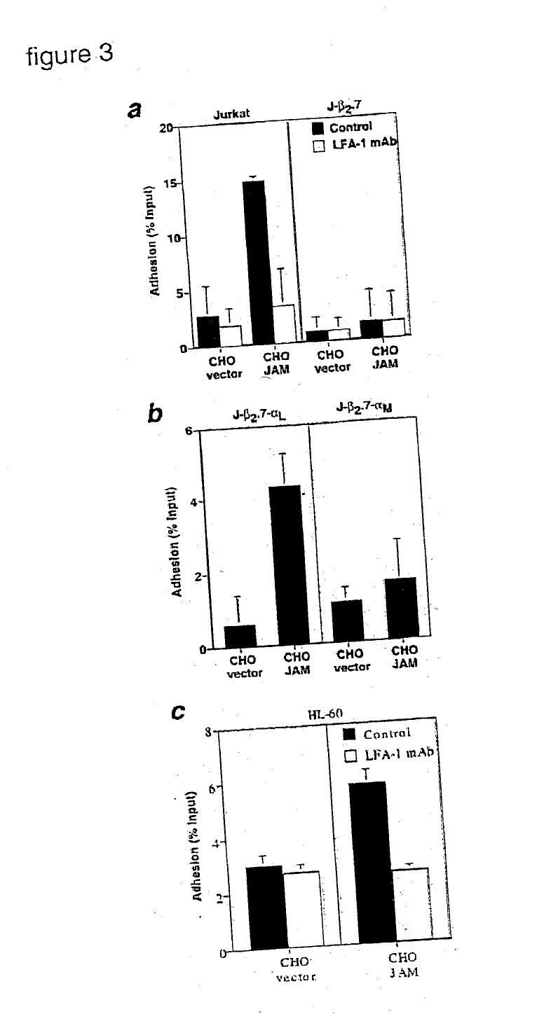

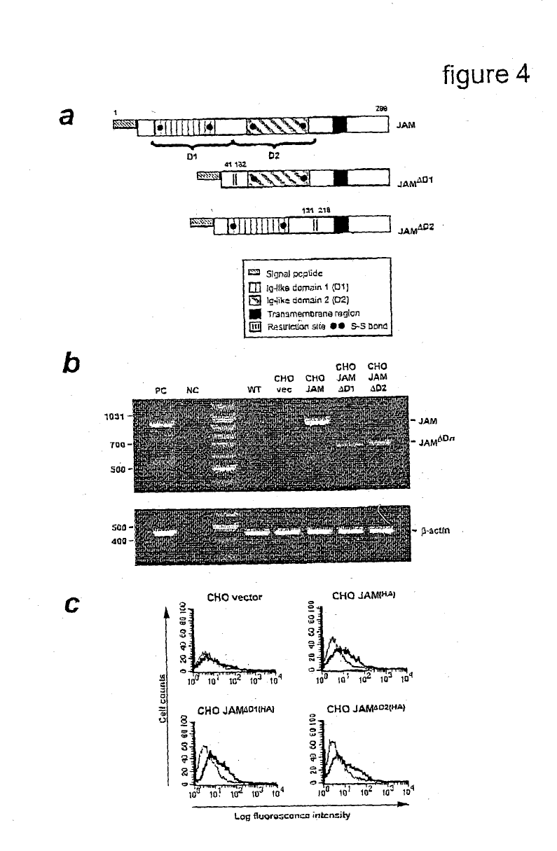

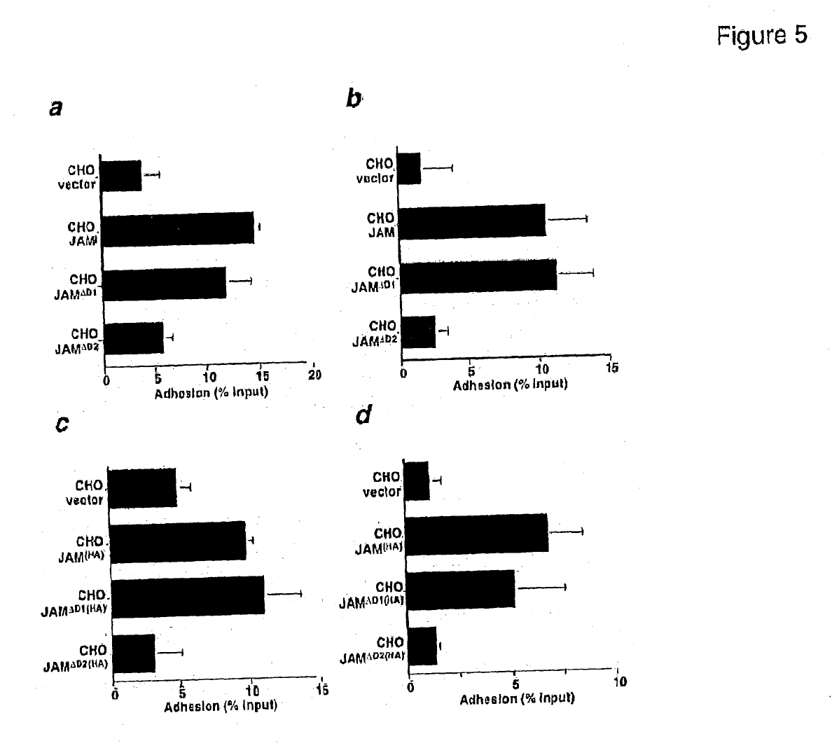

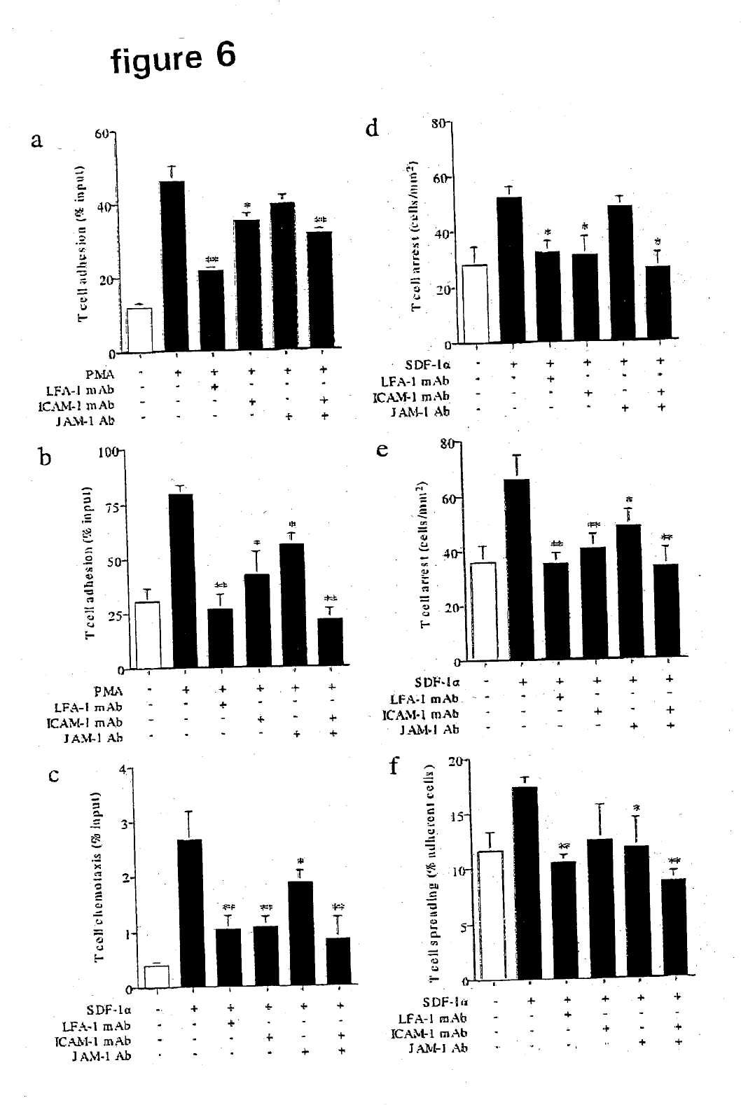

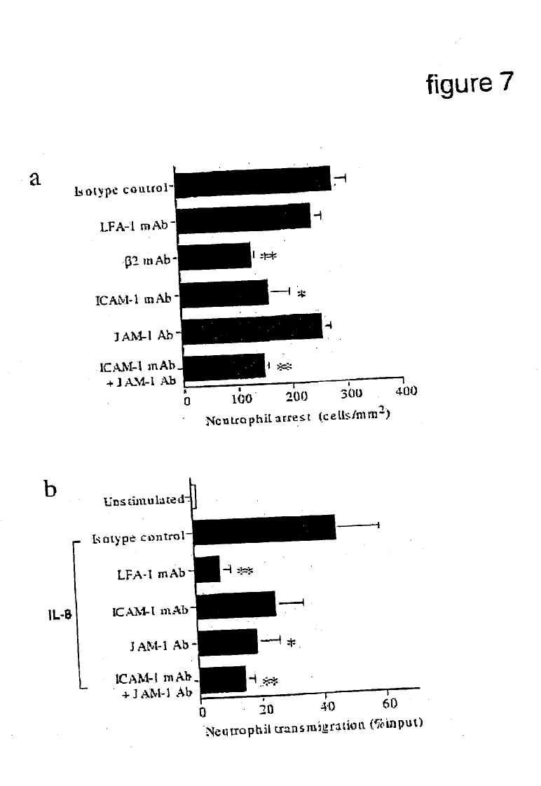

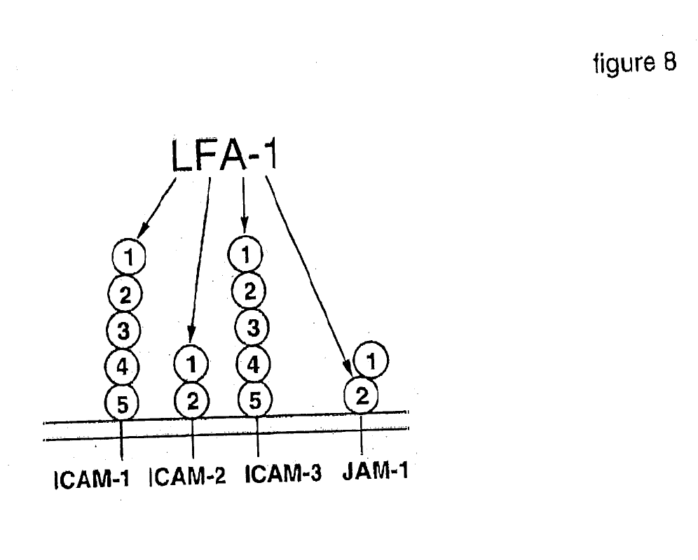

Using the yeast two-hybrid assay in order to identify specific interaction partners for the α_{L} chain cytoplasmic domain of LFA-1 (data not shown), we isolated a clone encoding the full-length sequence of human JAM-1 known previously to be a component of tight junctions (16,17). To confirm the interaction of LFA-1 and JAM-1 and to explore its functional consequences, we generated a cell model exhibiting JAM-1 surface expression, and analyzed JAM-1 expression on primary cells and cell lines. Chinese hamster ovary (CHO) cells, which do not express endogenous JAM-1, were transfected with JAM-1 or vector alone. Using murine polyclonal antibodies (Ab) raised against human JAM-1, we detected surface expression of JAM-1 on CHO-JAM transfectants but not CHO-vector transfectants by flow cytometry (FIG. 1a). Western blot analysis with the JAM-1 Ab revealed expression of JAM-1 in membrane fractions but not in cytosolic fractions of CHO-JAM transfectants (FIG. 1b). The JAM-1 Ab failed to recognize wildtype CHO cells, consistent with their lack of JAM-1 expression (FIG. 1a,b). Flow cytometry revealed that JAM-1 is expressed to a variable extent on the surface of human umbilical vein endothelial cells (HUVEC), epithelial HeLa cells, and leukocytes, for example monocytic Mono Mac 6, Jurkat and J-(2.7 T lymphoid cells, CD4⁺CD45RO⁺ T cells and neutrophils but hardly detectable on myeloid HL-60 cells (FIG. 1c and data not shown), in accordance with previous findings (16,17).

2.2 JAM-1 is a LFA-1 Ligand Supporting Leukocyte Adhesion

We tested if JAM-1 can interact with LFA-1 and may thereby be a substrate for leukocyte adhesion. To study potential interactions between LFA-1 and JAM-1, we performed static leukocyte adhesion assays on adherent CHO cells. Following stimulation with phorbol-12-myristate-13-acetate (PMA) or the chemokine stromal-derived factor (SDF)-1α, Jurkat T cells expressing LFA-1 exhibited substantial binding to CHO-JAM transfectants but not to CHO-vector transfectants or wild-type cells (FIG. 2a, data not shown). Pre-incubation of Jurkat cells with TS1/18, a blocking β₂ mAb, or pre-incubation of CHO transfectants with the JAM-1 Ab reduced adhesion to background levels, indicating that this binding was specifically mediated by LFA-1 and JAM-1 (FIG. 2a). Pretreatment of Jurkat cells with JAM-1 Ab had no effect on adhesion (19.0+3.6% versus 15.2±1.5% in control cells), thus excluding homophilic interactions of JAM-1. Conversely, adhesion assays on immobilized LFA-1 (retained in an active form and without co-purified JAM-1 detectable at concentrations used for coating, (FIG. 2b) clearly revealed that only CHO-JAM but not CHO-vector transfectants substantially bound to LFA-1 (FIG. 2c). Again, pre-incubation with TS1/18 or JAM-1 Ab completely inhibited the specific adhesion of CHO-JAM transfectants to immobilized LFA-1 (FIG. 2c). Our results consistently indicate that cell surface-expressed JAM-1 functions as a specific ligand for LFA-1 in cell-cell adhesion.

2.3 Adhesive Interactions with JAM-1 are Specific for LFA-1

To confirm that the adhesion of Jurkat cells to CHO-JAM transfectants was specific for LFA-1, static adhesion assays were performed using the mutant Jurkat J-β₂.7 cells, which lack LFA-1 surface expression due to a deficiency in α_{L} (25). In contrast to wild-type Jurkat cells, PMA-stimulated J-β₂.7 cells did not exhibit adhesion to CHO-JAM transfectants (FIG. 3a). Reconstitution of LFA-1 surface expression in J-β₂.7 cells by transfection with α_{L} cDNA (26) restored adhesion to CHO-JAM but not CHO-vector transfectants (FIG. 3b), indicating that adhesion of Jurkat calls to CHO-JAM transfectants requires the presence of intact surface-expressed LFA-1. In contrast, expression of the β₂-integrin Mac-1 in J-β₂. 7 cells by transfection with α_{M} cDNA did not confer binding to CHO-JAM cells (FIG. 3b); however, consistent with previous findings (26), this resulted in robust Mac-1-dependent adhesion after PMA stimulation on immobilized ICAM-1 (data not shown), indicating functional Mac-1. Together, this reveals that JAM-1 specifically interacts with LFA-1 but not Mac-1.

Since both LFA-1-bearing Jurkat and al-deficient J-β₂.7 cells show equivalent JAM-1 surface expression (FIG. 1c), the remarkable difference in their adhesion indicates that homophilic interactions of JAM-1 do not contribute to leukocyte adhesion on CHO-JAM transfectants. Leukocyte adhesion due to homophilic JAM-1 interactions was also ruled out by experiments using myeloid HL-60 cells which lack detectable JAM-1 surface expression but exhibited LFA-1-dependent adhesion to CHO-JAM transfectants (FIG. 3c).

Moreover, the binding of CHO-JAM cells in suspension to adherent CHO-JAM cells did not differ from that of wild-type CHO cells (data not shown). Thus, homophilic interactions of surface JAM-1 appear to be insufficient to support adhesion, in accordance with findings that recombinant solid phase JAM-1 failed to mediate adhesion of JAM-1-transfected CGS cells (17).

2.4 Binding to LFA-1 Requires Domain 2 of JAM-1

To characterize which domain(s) of JAM-1 were responsible for the interaction with LFA-1, we performed a deletional analysis by construction of JAM-1 mutants deficient in either the Ig-like domain 1 or 2 (FIG. 4a). The efficient recognition of JAM-1 by the JAM-1 Ab appeared to require full-length JAM-1, possibly by recognition of an epitope encompassing both domain 1 and 2 (data not shown). Thus, we detected the expression of wild-type JAM-1 and JAM-1 deletion mutants in CHO cells transfected with constructs which lacked an epitope tag by RT-PCR (FIG. 4b). Similar results were obtained for JAM-1 constructs that were tagged with an hemagglutinin (HA) epitope tagged adjoined- to the C-terminal sequence (data not shown). In addition, flow cytometric analysis of permeabilized cells (FIG. 4c) and western blot analysis of membrane fractions (not shown) with a polyclonal HA Ab confirmed equivalent surface membrane expression for all constructs. Both transfectants with or without HA epitope tag were subsequently used as a substrate for adhesion assays with PMA-stimulated Jurkat cells (FIG. 5a,c). CHO cells expressing JAM-1 deficient in the N-terminal domain 1 (CHO-JAM^{ΔD1}) supported considerable adhesion of Jurkat cells, equivalent to that seen on CHO cells expressing full-length JAM-1 (FIG. 5a,c). In contrast, the adhesion of Jurkat cells on CHO transfectants expressing JAM-1 lacking the membrane-proximal domain 2 (CHO-JAM^{ΔD2}) did not differ from background binding on CHO-vector transfectants (FIG. 5a,c). Like CHO cells expressing full-length JAM-1, CHO-JAM^{ΔD1} transfectants exhibited substantial adhesion to immobilized purified LFA-1, whereas binding of CHO-JAM^{ΔD2} transfectants to LFA-1 did not differ from background binding of CHO-vector transfectants (FIG. 5b,d). In particular when adjusted for the amount of protein expression, the cytoplasmic HA epitope tag did not appear to alter binding characteristics of the CHO transfectants. Thus, our results indicate that the membrane-proximal domain 2 of JAM-1 is crucial and sufficient for the direct interaction with LFA-1 and suggest a role of JAM-1 in leukocyte adhesion and recruitment.

2.5 Crucial Role of JAM-1 in Memory T Cell Transmigration

To elucidate the functional and mechanistic relevance of LFA-1-JAM-1 interactions in a more physiologic context, we performed adhesion and transmigration assays using human umbilical vein endothelial cells (HUVEC) and isolated CD4⁺CD45RO⁺ memory T lymphocytes which express the SDF-1α receptor, CXCR4, crucial for their inflammatory recruitment (27). As shown by inhibition with blocking Ab, PMA-induced adhesion and SDF-1α-triggered transendothelial chemotaxis of CD4⁺CD45RO⁺ T cells on HUVEC left untreated or co-stimulated with the inflammatory cytokines tumor necrosis factor-α (TNF-α) and interferon-γ (IFN-γ) was largely dependent on LFA-1 (FIG. 6a-c). In contrast, the contribution of the LFA-1 ligand ICAM-1 to adhesion was less pronounced (FIG. 6a-b), consistent 10′ with previous findings (1,6,8-10) suggesting an involvement of additional LFA-1 ligands Notably, the JAM-1 Ab did not affect PMA-stimulated adhesion of CD4⁺CD45RO⁺ T cells on resting HUVEC, either alone or when combined with an ICAM-1 mAb, but impaired transendothelial migration (FIG. 6a,c). On HUVEC treated with a combination of TNF-α and IFN-γ to induce a redistribution of JAM-1 from the junctions to the endothelial surface, (16) (not shown), JAM-1 Ab alone reduced binding of CD4⁺CD45RO⁺ T cells (FIG. 6b). Combined use of the JAM-1 Ab with an ICAM-1 mAb additively inhibited adhesion of CD4⁺CD45RO⁺ T cells to a similar extent as seen using a LFA-1 mAb (FIG. 6b). Experiments on cytokine-stimulated HUVEC were performed in the presence of a VLA-4 mAb to prevent interactions with VCAM-1 expressed following cytokine activation, which have been identified in the stable arrest of CD4⁺ T cells on TNF-α-activated HUVEC (also under conditions of flow) (28). Despite a redundancy of receptor-ligand pairs, this allowed us to detect a dependence for LFA-1- and JAM-1 in leukocyte transmigration. Similar results were obtained when adhesion was stimulated with SDF-1α (data not shown).

These data were further strengthened by adhesion and transmigration assays on inflamed endothelia under physiologic flow conditions which were performed in the presence of a VLA-4 mAb to exclude interactions with VCAM-1 or other VLA-4 ligands and to enable demonstration of LFA-1 and JAM-1 functions. The arrest of CD4⁺CD45RO⁺ T cells in flow was triggered by SDF-1α added and immobilized on TNF-α-stimulated HUVEC (FIG. 6d), as has been described for CD34⁺ cells (29). Inhibition with LFA-1 or ICAM-1 mAb revealed that the arrest of these cells was dependent on LFA-1 and mediated by its ligand ICAM-1 (FIG. 6d). Again, the JAM-1 Ab did not affect the LFA-1-dependent arrest of memory T-cells (FIG. 6d), however blocking JAM-1 attenuated subsequent spreading and transmigration (data not shown). In contrast, on HUVEC co-stimulated with TNF-α and IFN-γ to induce JAM-1 redistribution, the JAM-1 Ab and ICAM-1 mAb alone reduced arrest and, when combined, appeared to have an additive effect (FIG. 6e). The JAM-1 Ab also impaired the shape change of CD4⁺CD45RO⁺ T cells, indicative of spreading or transmigration, and in combination with the ICAM-1 mAb produced an additive inhibition of shape change (FIG. 6f). Our data suggest that JAM-1 crucially participates in sequential steps of leukocyte recruitment and cooperates with ICAM-1 as an alternative ligand for LFA-1, playing an integral role in LFA-1-dependent transmigration of memory T cells, but also contributing to their adhesion under specific conditions of inflammatory stimulation.

2.6 Involvement of JAM-1 in Neutrophil Transmigration

Since JAM-1 has previously been implicated in the transmigration of myeloid cells (15), we extended our analysis of the involvement of JAM-1 in leukocyte recruitment to neutrophils. In contrast to results with memory T cells, JAM-1 was less important for CXCR2-mediated arrest (data not shown) of neutrophils on HUVEC costimulated with TNF-α and IFN-γ in shear flow (FIG. 7a). This result was in accordance with blocking data suggesting that LFA-1 alone was not sufficient to alter arrest of neutrophils, while the combined blockade of LFA-1 and Mac-1 by a 92 mAb resulted in significant inhibition of neutrophil arrest (FIG. 7a). These findings confirmed a report showing that both LFA-1 and Mac-1 are redundantly involved in arrest (30). Thus, the lesser contribution of LFA-1 and its ligand JAM-1 in neutrophils, as opposed to its prominent role in memory T cell arrest, appears due to the involvement of Mac-1. In contrast, neutrophil arrest was inhibited by blocking ICAM-1 (FIG. 7a), which serves as a ligand for both LFA-1 and Mac-1, further suggesting that binding to JAM-1 is specific for LFA-1 and not Mac-1. In contrast, the transendothelial chemotaxis of neutrophils in response to interleukin-8 (IL-8) was largely dependent on LFA-i and inhibited by blocking CAM-1 (FIG. 7b). Notably, pretreatment of HUVEC with the blocking JAM-1 Ab alone or in combination with the ICAM-1 mAb attenuated neutrophil transmigration (FIG. 7b). Similar findings were obtained for chemokine-induced transmigration of monocytes, in accordance with previous findings (15). Hence, JAM-1 is crucially involved in the transmigration of both myelomonocytic, cells, as well as lymphocytes.

3. Discussion

We have found that JAM-1 is a specific ligand for the 2 integrin LFA-1, providing the first evidence for a heterotypic adhesive interaction of JAM-1 with a leukocytic counter-receptor. The membrane-proximal Ig-like domain 2 of JAM-1 was necessary, and sufficient for this interaction, whereas the N-terminal domain 1 was not directly involved. The binding of LFA-1 to JAM-1 contributed to the PMA- and chemokine-triggered adhesion and transendothelial migration of memory T cells, This can occur in cooperation with ICAM-1, and may be particularly relevant under physiologic flow conditions, and after cytokine activation of the endothelium resulting in JAM-1 redistribution. Moreover, JAM-1 crucially participated in chemokine-induced transendothelial migration of neutrophils.

Junctional adhesion molecules have recently emerged as a distinct group of IgSF proteins. Originally described as platelet F11 receptor on human cells (31,32), the structure and function of murine JAM-1 was subsequently characterized (15) and found to be identical to antigen 106 (21). Additional members have since been identified, for example human JAM-2 (33) also known as human vascular endothelial junction-associated molecule (VE-JAM) (34), murine JAM-2 (35,36), and murine JAM-3 (35) also known as murine VE-JAM (34). Since human JAM-2 is homologous to murine JAM-3, but not to murine JAM-2, this nomenclature may require some redesignation. Different functions of JAM-1 described so far remain to be fully elucidated: (1) its involvement in the organization of, tight junctions at adherent cell-cell contacts (15-20), (2) its role in human hematopoietic cells, for example in platelet activation and aggregation following cross-linking by a stimulatory JAM-1-Ab (31-32), and in particular (3) its contribution to leukocyte adhesion and: transmigration across endothelium or epithelium during inflammatory leukocyte recruitment (15-23).

In endothelial or epithelial cells, JAM-1 is selectively concentrated at the apical region of intercellular junctions and participates as a component of the tight junctional complex by association with multiple proteins, for instance AF-6, ZO-1, cingulin or occludin, which may contribute to distinct states of junctional maturation or function, and enable anchoring of JAM-1 to the cytoskeleton (19,37), However, its expression at areas of intercellular contact can also occur independently of pre-organized tight junctions and their protein associations (15,19). Here, the localization of JAM-1 is thought to be triggered by extracellular homophilic interactions (18, 20) on adjacent cells. The combined treatment with the pro-inflammatory cytokines TNF-α and IFN-γ caused a dissociation of JAM-1 from intercellular junctions and redistribution to the endothelial surface, infering a regulatory function in junctional integrity and permeability (16). Thus, it could be envisioned that such mechanisms are permissive for leukocyte transmigration. Previous studies have indeed involved JAM-1 in the transendothelial migration. The murine JAM-1 mAb BV11 blocked spontaneous or chemokine-induced transendothelial migration of murine monocytes (which do not express JAM-1) in vitro and reduced inflammatory infiltrates in a model of meningitis in vivo without affecting homophilic interactions of JAM-1 at junctions, as reflected by paracellular permeability of JAM-1 transfectant monolayers for dextran (15,23). The effects of BV11, however, may also be due to an inhibition of dimerization (54). On the other hand, a JAM-1 mAb that increased permeability did not reduce monocyte transmigration (55). Our data now reveal that leukocytic LFA-1 is a heterotypic counter-receptor for JAM-1.

In contrast to our data, soluble JAM-1 ectopically expressed in insect cells did not appear to support adhesion of lymphoid cells (34). This may be due to differences in the post-translational modification of JAM-1 for example glycosylation which is known to affect IgSF adhesive properties (11) and may occur at potential glycosylation sites in the LFA-1-binding domain 2, or due to a requirement of cell surface expression for correct orientation and function of JAM-1, but most likely due to the use of unstimulated cells which do not express activated LFA-1.

Although our study demonstrates that cell-surface expressed JAM-1 is a heterophilic ligand for LFA-1, the finding that monocyte adhesion to endothelial cells expressing JAM-1 was not affected by JAM-1 mAb BV11 (15) can be explained by a redundancy in adhesive receptor-ligand pairs or by insufficient surface expression when JAM-1 is not redistributed from the junctions. Indeed, on endothelium co-stimulated with TNF-α and IFN-γ to trigger JAM-1 redistribution, we found that JAM-1 contributed to LFA-1-dependent adhesion and shear-resistant arrest of memory T cells.

Notably, JAM-1 is also expressed in hematopoietic cells (17,21,22), however a role in platelet activation and aggregation is its only function in hematopoietic cells known so far (22,38). Our data provide several lines of evidence to corroborate the conclusion that homophilic interactions involving leukocyte JAM-1 are not crucial for the firm adhesion of leukocytes. 1: The surface expression of JAM-1 on wild-type Jurkat cells, α_{L}-deficient J-β₂.7 cells and HL-60 cells did not correlate with their adhesion to CHO-JAM transfectants. 2: Blocking leukocyte JAM-1 did not affect binding of T cells to CHO-JAM transfectants or HUVEC. (3) Suspended and adherent CHO-JAM cells did not show specific interactions These results are supported by findings that recombinant JAM-1 failed to mediate adhesion of JAM-1-transfected COS cells in solid phase assays, possibly due to low affinity, and consistent with a notion that homotypic interactions may arise preferably in regions of cell-cell contacts with sufficient JAM-11 density, for example interendothelial junctions (17). Although we did not find any evidence for homophilic interactions of JAM-1 between leukocytes and CHO-JAM transfectants or HUVEC, it cannot be ruled out that interactions of low stringency may occur during leukocyte recruitment. Indeed, it has been shown that CHO cells expressing JAM-1 facilitate adhesion of platelets (39), presumably via homophilic interactions, although other heterophilic receptors on platelets have not been excluded, for instance by blocking Ab. Alternatively, a lack of inhibitory effects on homophilic interactions by the JAM-1 Ab may be due to the notion that the epitope affected by the binding of JAM-1 Ab is likely to be located in the membrane-proximal domain 2 which binds LFA-1 rather than the N-terminal region of JAM-1 involved in homophilic interactions (18).

Functional assays with physiologic cell types indicated that JAM-1 plays an important role in LFA-1-dependent transendothelial migration of memory T cells in response to SDF-1α which is known To be involved in their inflammatory recruitment in vivo (27), Blocking JAM-1 reduced the transmigration of memory T cells both across HUVEC-coated transwell filters and on HUVEC under flow conditions, and in combination with ICAM-1 blockade additively inhibited migration in flow, infering that both JAM-1 and ICAM-1 are required as partially alternative ligands of LFA-1 to achieve optimal diapedesis. Notably, blocking LFA-1 had often-appeared to be more effective in inhibiting leukocyte transmigration than blocking any of its known endothelial ligands, for example ICAM-1. Hence, the interaction of LFA-1 with JAM-1 described here substantiates the hypothesis that LFA-1 may use or even require multiple junctional ligands during diapedesis. Moreover, the redistribution of JAM-1 to the endothelial surface under inflammatory conditions supported adhesion of memory T cells and facilitated subsequent transmigration which may not only be due to increased junctional permeability but also to a haptotactic gradient of JAM-1 guiding attached T cells to diapedese.

We found that both ICAM-1 and JAM-1-contributed to LFA-1-dependent transendothelial migration of neutrophils in response to IL-8. By revealing an involvement of both LFA-1 ligands in myeloid cell transmigration, this expands on results obtained for the participation of JAM-1 in chemokine-induced transmigration of monocytes (15). In contrast to T cells, blocking JAM-1 was not sufficient to inhibit CXCR2-mediated neutrophil arrest on HUVEC costimulated with TNF-α and IFN-γ in shear flow. This is likely due to the redundant involvement of other receptor-ligand pairs, e.g. Mac-1 and ICAM-1 (30). Thus, while JAM-1 may facilitate adhesion of T cells but not neutrophils, it may play a more prominent role in transendothelial diapedesis of myeloid cells.

Our results parallel a report showing that the IgSF protein PECAM-1 which engages in homophilic associations also functions as a ligand for αᵥβ₃ integrin (14). Like JAM-1, PECAM-1 that is localized juxtaposed to tight junctions albeit less apical (40) is redistributed from cell-cell contacts following stimulation with TNF-α and IFN-γ and plays a role in extravasation (16,24).

This underscores the concept that IgSF members which-can interact by homephilic binding may be particularly suited to support transendothelial migration of leukocytes. It has been suggested that the homophilic association of JAM-1 relevant for dimerization involves the N-terminal region of JAM-1 (18). A, recent model based on the crystallographic structure of recombinant soluble JAM has proposed that U-shaped JAM dimers (interacting via a N-terminal motif) are oriented in a cis-configuration on the cell surface and form a two-dimensional network by trans-interactions of the N-terminal domains in a common central plane with dimers from an opposite cell surface (41). Notably, we found that LFA-1 binds to the membrane-proximal domain 2 of JAM-1 which, according to this model, protrudes almost perpendicular from the cell surfaces. Thus, it would be intriguing to speculate that beyond a primary adhesive interaction with JAM-1, leukocyte LFA-1 may serve to intercept JAM-JAM interactions at interendothelial junctions during transmigration. The finding that blocking both LFA-1 and JAM-1 did not cause additive effects suggests that homophilic interactions of JAM-1 between T cells and endothelial cells per se are not critical to fully accomplish transmigration. However, it is conceivable that homophilic interactions of JAM-1 via the N-terminal domain may still occur following the binding of is LFA-1 to domain 2 to successively capture opposing JAM-1 dimers on the leukocyte and in turn to recruit endothelial JAM-1 as an interaction partner for LFA-1 This scenario would be especially challenging in light of our intial discovery that the interaction of LFA-1 and, JAM-1 occurs via the cytoplasmic domains. This interaction is indicative of a close proximity of LFA-1 and JAM-1 on leukocytes, and may be highly regulated by cellular activation, so that it may not be constantly detectable by copurification. Thus, a complex interplay of heterophilic binding of LFA-1 to JAM-1 and homophilic trans-interactions of JAM-1 may provide a molecular ‘zipper’ for transmigration.

In conclusion, we have found that JAM-I serves as a novel ligand for the integrin LFA-1. Hence, these data provide the first evidence for a heterophilic interaction of LFA-1 with an IgSF ligand that does not belong to the ICAM subgroup. Moreover, this constitutes the first interaction of LFA-1 that appears to mainly require the membrane-proximal but not the N-terminal domain 1 of an IgSF member as the primary recognition site (1,42). The binding of LFA-1 on leukocytes to JAM-1 at the interendothelial or interepithelial junctions or on the endothelial surface following inflammatory stimulation may support the adhesion of leukocytes and may disrupt or intercalate homophilic junctional JAM-1 interactions, thereby unlocking intercellular junctions and guiding leukocytes during transmigration. Future studies may focus on the intricate cross-talk between homophilic and heterophilic interactions of JAM-1 in leukocyte transmigration, as well as on putative cellular signaling pathways via the cytoplasmic domain of JAM-1 and the identification of additional interaction partners for other JAM family proteins.

The use of effectors capable of modulating interaction between JAM-1 and LFA-1, particularly effectors capable of blocking inflammatory leukocyte recruitment has enormous therapeutic potential for the selective treatment of inflammatory and infectious disorders. Examples of such disorders are vascular inflammatory disorders, such as artherosclerosis, arteritis etc., autoimmune and connective tissue diseases, such as rheumathoid arthritis, diseases of the central nervous system, such as meningitis, multiple sclerosis, due to JAM expression on brain microvascular endothelial cells or inflammatory bowel disease, such as ulcerative colitis, Crohn's disease due to JAM-1 expression on intestinal epithelial cells.

### REFERENCES

1. Springer, T. A. Traffic, signals for lymphocyte recirculation and leukocyte emigration: the multistep paradigm. Cell 76, 301-314 (1994).
2. Weber, C., Kitayama, J. & Springer, T. A. Differential regulation of 11 and β₂ integrin avidity by chemoattractants in eosinophils. Proc. Natl, Acad. Sci. USA 93, 10939-10944 (1996).
3. Campbell, J. J. et al. Chemokines and the arrest of lymphocytes rolling under flow conditions. Science 279, 381-384 (1998).
4, Smith, C. W., Marlin, S. D., Rothlein, R., Toman, C. & Anderson, D. C. Cooperative interactions of LFA-1 and Mac-1 with intercellular adhesion molecule-1 in facilitating adherence and transendothelial migration of human neutrophils in vitro. J. Clin. Invest. 83, 2008-2017 (1989).
5. Weber, C., Lu, C. F., Casasnovas, J. M. & Springer, T. A. Role of α_{L}β₂ integrin avidity in transendothelial chemotaxis of mononuclear cells. J. Immunol. 159, 3968-3975 (1997).
6. Rothlein, R., Dustin, M. L., Marlin, S. D. & Springer, T. A. A human intercellular adhesion molecule (ICAM-1) distinct from LFA-1. J. Immunol. 137, 1270-1274 (1986).
7. Marlin, S. D. & Springer, T. A. Purified intercellular adhesion molecule-1 (ICAM-1) is a ligand for lymphocyte function-associated antigen, 1 (LFA-1). Cell 51, 813-819 (1987).
8. Nortamo, P., Salcedo, R., Timonen, T., Patarroyo, M. & Gahmberg, C. G. A monoclonal antibody to the human leukocyte adhesion molecule intercellular adhesion molecule-2. Cellular distribution and molecular characterization of the antigen. J. Immunol. 146, 2530-2535 (1991).
9. de Fougerolles, A. R. & Springer, T. A. Intercellular adhesion molecule 3, a third adhesion counter-receptor for lymphocyte function-associated molecule 1 on resting lymphocytes. J. Exp. Med. 175, 185-190 (1992).
10. Bleijs, D. A, Binnerts, M. E., van Viet, S. J., Figdor, C. G. & van Kooyk, Y. Low-affinity LFA-1/ICAM-3 interactions augment LFA-1/ICAM-1-mediated T cell adhesion and signaling by redistribution of LFA-1. J. Cell Science 113, 391-400 (2000).
11. Diamond, M. S., Staunton, D. E., Marlin, S. D. & Springer, T. A. Binding of the integrin Mac-1 (CD11 b/CD18) to the third immunoglobulin-like domain of ICAM-1 (CD54) and its regulation by glycosylation. Cell 65, 961-971 (1991).
12. Elices, M. J. et al. VCAM-1 on activated endothelium interacts with the leukocyte integrin VLA-4 at a site distinct from the VLA-4/fibronectin binding site. Cell 60, 577-584 (1990).
13. Berlin, C. et al. 97 integrin mediates lymphocyte binding to the mucosal vascular addressin MAdCAM-1. Cell 74, 185-185 (1993).
14. Piali, L. et al. CD31/PECAM-1 is a ligand for αᵥβ₃ integrin involved in adhesion of leukocytes to endothelium. J. Cell Biol. 130, 451-460 (1995).
15. Martin-Padura, I. et al. Junctional adhesion molecule, a novel member of the immunoglobulin superfamily that distributes at intercellular junctions and modulates monocyte transmigration. J. Cell Biol. 142, 117-127 (1998).
16. Ozaki, H. et al. Combined treatment of TNF-α and IFN-γ causes redistribution of junctional adhesion molecule in human endothelial cells. J. Immunol. 163, 553-557 (1999).
17. Williams, L. A., Martin-Padura, Il, Dejana, E., Hogg, N. & Simmons, D. L. Identification and characterisation of human junctional adhesion molecule (JAM), Mol. Immunol. 36, 1175-1188 (1999).
18. Bazzoni, G. et al. Homophilic interaction of junctional adhesion molecule. J. Biol. Chem. 275, 30970-30976 (2000).
19. Ebnet, K., Schulz, C. U., Meyer, Z. B. M., Pendl, G. G. & Vestweber, D. Junctional adhesion molecule interacts with the PDZ domain-containing proteins AF-6 and Z0-1 J. Biol. Chem. 275, 27979-27988 (2000).
20. Liang, T. W. et al. Characterization of huJAM: evidence for involvement in cell-cell contact and tight junction regulation. Am. J. Physiol. Cell Physiol. 279, C1733-C1743 (2000).
21. Malergue, F. et al. A novel immunoglobulin superfamily junctional molecule expressed by antigen presenting cells, endothelial cells and platelets. Mol. Immunol. 35, 1111-1119 (1998).
22. Sobocka, M. B. et al. Cloning of the human platelet F11 receptor: a cell adhesion molecule member of the immunoglobulin superfamily involved in platelet aggregation. Blood 95, 2600-2609 (2000).
23. Del Maschio, A. et al. Leukocyte recruitment in the cerebrospinal fluid of mice with experimental meningitis is inhibited by an antibody to junctional adhesion molecule (JAM). J. Exp. Med. 190, 1351-1356 (1999).
24. Muller, W. A., Weigl, S. A., Deng, X. & Phillips, D. M. PECAM-1 is required for transendothelial migration of leukocytes. J. Exp. Med. 178, 449-460 (1993).
25. Weber, K. S., York, M. R., Springer, T. A. & Klickstein, L. B. Characterization of lymphocyte function-associated antigen 1 (LFA-1)-deficient T cell lines: the αLβ₂ subunits are interdependent for cell surface expression. J. Immunol. 158, 273-279 (1997).
26. Weber, K. S., Klickstein, L. B. &-Weber, C. Specific activation of leukocyte β₂ integrins lymphocyte function-associated antigen-1 and Mac-1 by chemokines mediated by distinct pathways via the α subunit cytoplasmic domains, Mol. Biol. Cell 10, 861-873 (1999).
27. Nanki, T. et al. Stromal cell-derived factor-1-CXC chemokine receptor 4 interactions play a central role in CD4⁺ T cell accumulation in rheumatoid arthritis synovium. J. Immunol. 165, 6590-6598 (2000).
28. Luscinskas, F. W., Ding, H. & Lichtman, A. H. P-selectin and vascular cell adhesion molecule 1 mediate rolling and arrest, respectively, of CD4⁺ T lymphocytes on tumor necrosis factor α-activated vascular endothelium under flow. J. Exp. Med. 181, 1179-1186 (1995).
29, Peled, A. et al. The chemokine SDF-1 stimulates integrin-mediated arrest of CD34⁺ cells on vascular endothelium under shear flow. J. Clin. Invest. 104, 1199-1121 (1999).
30. Gopalan, P. K. et al. Preferential sites for stationary adhesion of neutrophils to cytokine-stimulated HUVEC under flow conditions. J. Leukoc. Biol. 68,47-51 (2000).
31. Kornecki, E., Walkowiak, B., Naik, U. P. & Ehrlich, Y. H. Activation of human platelets by a stimulatory monoclonal antibody. J. Biol. Chem. 265, 10042-10048 (1990).
32. Naik, U. P., Ehrlich, Y. H. & Kornecki, E. Mechanisms of platelet activation by a stimulatory antibody: cross-linking of a novel platelet receptor for monoclonal antibody F11 with the FcγRII receptor. Biochem. J. 310, 155-162 (1995).
33. Cunningham, S. A. et al. A novel protein with homology to the junctional adhesion molecule. Characterization of leukocyte interactions. J. Biol. Chem. 275, 34750-34756 (2000).
34. Palmeri, D., van Zante, A., Huang, C. C., Hemmerich, S. & Rosen, S. D. Vascular endothelial junction-associated molecule, a novel member of the immunoglobulin superfamily, is localized to intercellular boundaries of endothelial cells. J. Biol. Chem. 275, 19139-19145 (2000).
35. Aurrand-Lions, M. A., Duncan, L., Du, P. L. & Imhof, B. A. Cloning of JAM-2 and JAM-3: an emerging junctional adhesion molecular family? Curr. Top. Microbiol. Immunol. 251, 91-98 (2000).
36. Aurrand-Lions, M. A., Duncan, L., Ballestrem, C. & Imhof, B. A. JAM-2, a novel immunoglobulin superfamily molecule, expressed by endothelial and, lymphatic cells. J. Biol, Chem. 276, 2733-2741 (2001).
37. Bazzoni, G. et al. Interaction of junctional adhesion molecule with the tight junction components ZO-1, cingulin, and occludin. J. Biol. Chem. 275, 20520-20526 (2000).
38. Ozaki, H. et al. Junctional adhesion molecule (JAM) is phosphorylated by protein kinase C upon platelet activation. Biochem. Biophys. Res. Commun. 276, 873-878 (2000).
39. Naik, U. P., Naik, M. U., Eckfeld, K., Martin-DeLeon, P. & Spychala, J. Characterization and chromosomal localization of JAM-1, a platelet receptor for a stimulatory monoclonal antibody. J. Cell Sci. 114, 539-547 (2001).
40. Ayalon, O., Sabanai, H., Lampugnani, M. G., Dejana, E. & Geiger, B. Spatial and temporal relationships between cadherins and PECAM-1 in cell-cell junctions of human endothelial cells. J. Cell Biol. 126, 247-258 (1994).
41. Kostrewa, D. et al. X-ray structure of junctional adhesion molecule: structural basis for homophilic adhesion via a novel dimerization motif. EMBO. 20, 4391-4398 (2001).
42. Klickstein, L. B., York, M. R., Fougerolles, A. R. & Springer, T. A. Localization of the binding site on intercellular adhesion molecule-3 (ICAM-3) for lymphocyte function-associated antigen 1 (LFA-1). J. Biol. Chem. 271, 23920-23927 (1996).
43. Horton, R. M., Hunt, H. D., Ho, S. N., Pullen, J. K. & Pease L. R, Engineering hybrid genes without the use of restriction enzymes: gene splicing by overlap extension. Gene 77, 61-68 (1989).
44. Weber, K. S., von Hundelshausen, P., Clark-Lewis, I., Weber, P. C. & Weber C. Differential immobilization and hierarchical involvement of chemokines in monocyte arrest and transmigration on inflamed endothelium in shear flow. Eur. J. Immunol. 29, 700-712 (1999).
45. Weber, C. et al. Specialized roles of the chemokine receptors CCR1 and CCR5 in the recruitment of monocytes and T_{H}1-like/CD45 RO⁺ T cells. Blood 97, 1144-1146 (2001).
46. Davis, H. L., Michel, M. L. & Whalen, R. G. Use of plasmid DNA for direct gene transfer and immunization. Ann. N.Y. Acad, Sci. 772, 21-29 (1995).
47. Weber, C., Alon, R., Moser, B. & Springer, T. A. Sequential regulation of α₄β₂ and α₅β₁ integrin avidity by CC chemokines in monocytes: implications for transendothelial chemotaxis. J. Cell Biol. 134, 1063-1073 (1996).
48. Kukreti, S., Konstantopoulos, K., Smith, C. W. & McIntire, L. V. Molecular mechanisms of monocyte adhesion to interleukin-1β-stimulated endothelial cells under physiologic flow conditions. Blood 89, 4104-4111 (1997).
49. Kompella, U. B. & Lee, V. H. Delivery systems for penetration enhancement of peptide and protein drugs: design considerations. Adv. Drug Deliv. Rev. 46, 211-245 (200.1).,
50. MacLachlan, I., Cullis, P. & Graham, R. W. Progress towards a synthetic virus for systemic gene delivery. Curr. Opin. Mol. Ther. 1, 252-259 (1999),
51. Liu, F. & Huang, L. Development of non-viral vectors for systemic gene delivery. J. Control Release 78, 259-266 (2002,
52. Coller, B. S. Anti-GPIIb/IIIa drugs: current strategies and future directions. Thromb. Haemost. 86, 427-443 (2001).
53. Boushey, H. A., Jr. Experiences with monoclonal antibody therapy for allergic asthma. J. Allergy Clin. Immunol. 108 (2 Suppl.), 577-83-(2001).
54. Bazzoni, G. et al. Homophilic interaction of junctional adhesion molecule. J. Biol, Chem. 275, 30970-30976 (2000).
55. Shaw, S. K. et al. Reduced expression of junctional adhesion molecule and platelet/endothelial cell adhesion molecule-1 (CD31) at human vascular endothelial junctions by cytokines tumor necrosis factor-alpha plus interferon-gamma does not reduce leukocyte transmigration under flow. Am. J. Pathol. 159, 2281-2291 (2001).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosure[s] of all applications, patents and publications, cited herein and of U.S. Provisional Application Ser. No. 60/371,734, filed Apr. 12, 2002 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method of monitoring or modulating lymphocyte function-associated antigen-1 (LFA-1) mediated processes or disorders comprising contacting a cell or an organism, which expresses LFA-1 with (i) a polypeptide selected from junctional adhesion molecule-1 (JAM-1) or an active fragment thereof, (ii) a nucleic acid molecule encoding the polypeptide of (i) or (iii) an effector of the polypeptide of (i) or the nucleic acid of (ii).

2. The method of claim 1 wherein the processes or disorders comprise a cell migration.

3. The method of claim 2 wherein the cell migration is a transendothelial migration of leukocytes.

4. The method of claim 3 wherein the leukocytes are selected from T cells and neutrophils.

5. The method of claim 1 wherein the processes or disorders comprise an arrest of T cells.

6. The method of claim 5 wherein the T cell arrest is triggered by 25 chemokines.

7. The method of claim 1 for the diagnosis, prevention or treatment of infectious or inflammatory disorders.

8. The method of claim 7 wherein the disorders are selected from the group consisting of vascular inflammatory disorders such as artherosclerosis, arteritis or reperfusion injury, autoimmune and connective tissue disorders such as rheumatoid arthritis, disorders of the nervous system such as meningitis or multiple sclerosis and inflammatory bowel disorders such as ulcerative colitis and Crohn's disease.

9. The method of claim 7 or 8 wherein the contacting comprises administering a pharmaceutical composition comprising an effective amount of (i), (ii) or (iii) and at least one physiologically acceptable carrier, diluent or adjuvant to a subject in need thereof.

10. The method of claim 9 wherein the composition is a therapeutic composition.

11. The method of claim 9 wherein the composition is a diagnostic 15 composition.

12. The method of claim 9 wherein the subject is a human.

13. The method of claim 1 for use in a screening system.

14. The method of claim 1 wherein the polypeptide (i) is a mammalian JAM-1, preferably selected from human JAM-1 (GenBank Accession No. AF1 72398) or an active fragment thereof.

15. The method of claim 14 wherein the active fragment comprises the membrane-proximal domain 2 of JAM-1 or a portion thereof.

16. The method of claim 15 wherein the active fragment comprises amino acids 125-212 of JAM-1 or a portion thereof, particularly portions adjacent to domain and/or the linker sequence 127-129.

17. The method of claim 1 wherein the nucleic acid (ii) encodes a mammalian JAM-1, preferably selected from human JAM-1 (Gen-Bank Accession No. AF1 72398) or an active fragment thereof.

18. The method of claim 1 wherein the effector (iii) is capable of modulating the interaction of JAM-1 and LFA-1.

19. The method of claim 18 wherein the effector (iii) interacts with JAM-1.

20. The method of claim 19 wherein the effector (iii) interacts with the membrane-proximal domain 2 of JAM-1.

21. The method of claim 18 wherein the effector (iii) interacts with LFA-1.

22. The method of claim 21 wherein the effector (iii) interacts with the inserted (I) domain of LFA-1.

23. The method of claim 1 wherein the effector (iii) is selected from antibodies and fragments thereof, biologically active nucleic acids, e.g. antisense molecules, RNA; molecules or ribozymes, peptides, peptide mimetics or scaffolds, and low-molecular weight organic compounds.

24. The method of claim 23 wherein the effector (iii) is an antibody which inhibits the transendothelial migration of human leukocytes, particularly neutrophils and memory T cells.

25. The method of claim 23 wherein the effector (iii) is a peptide, cyclic peptide, peptide mimetic or scaffold derived from the membrane-proximal domain 2 of JAM-1 or from the inserted (I) domain of LFA

26. A host cell or a non-human host organism capable of overexpressing JAM-1 and LFA-1 or active fragments thereof.

27. A cell-free assay system comprising a JAM-1 polypeptide and/or a LFA-1 polypeptide or active fragments thereof.

28. A method for identifying and/or characterizing the effect of compounds on LFA-1 mediated processes or disorders comprising:
(a) providing an assay system comprising
(i) a JAM-1 polypeptide or an active fragment thereof or a host cell or a nonhuman host organism capable of overexpressing JAM-1 or an active fragment thereof and
(ii) a LFA-1 polypeptide or an active fragment thereof or a host cell or a non-human host organism capable of overexpressing LFA-1, or an active fragment thereof,
(b) contacting the system of (a) with a compound to be tested and
(c) determining the effect of the compound on the interaction between (i) and (ii).


SEQUENCE LISTING (8 sequences)

SEQ ID NO: 1 - DNA, 33 bp, Artificial Sequence
  Description of Artificial Sequence primer
gcggggtaccatggggacaaaggcgcaagtcga

SEQ ID NO: 2 - DNA, 33 bp, Artificial Sequence
  Description of Artificial Sequence primer
gcggggatcctcacaccaggaatgacgaggtct

SEQ ID NO: 3 - DNA, 43 bp, Artificial Sequence
  Description of Artificial Sequence primer
gcggggtaccgccgccagccatggggacaaaggcgcaagtcga

SEQ ID NO: 4 - DNA, 48 bp, Artificial Sequence
  Description of Artificial Sequence primer
ggatgttaactgtaggcttggatggaggaattctgacttcaggttcag

SEQ ID NO: 5 - DNA, 48 bp, Artificial Sequence
  Description of Artificial Sequence primer
ttctgaacctgaagtcagaattcctccatccaagcctacagttaacat

SEQ ID NO: 6 - DNA, 47 bp, Artificial Sequence
  Description of Artificial Sequence primer
catttgaagtcatgggtgtcccataaggcacaagcacgatgagcttg

SEQ ID NO: 7 - DNA, 48 bp, Artificial Sequence
  Description of Artificial Sequence primer
ggtcaagctcatcgtgcttgtgccttatgggacacccatgacttcaaa

SEQ ID NO: 8 - DNA, 66 bp, Artificial Sequence
  Description of Artificial Sequence primer
gcggggatcctcaagcgtaatctggaacatcgtatgggtatccagccaccaggaatgacgaggtct